SEQUENCE LISTING (28 sequences)

SEQ ID NO: 1 - protein, 47 aa, Homo sapiens
ECCEKPLLEKSHCIAEVENDEMPADLPSLAADFVESKDVCKNYAEAK

SEQ ID NO: 2 - protein, 36 aa, Homo sapiens
VHTECCHGDLLECADDRADLAKYICENQDSISSKLK

SEQ ID NO: 3 - protein, 35 aa, Homo sapiens
RPCFSALEVDETYVPKEFNAETFTFHADICTLSEK

SEQ ID NO: 4 - protein, 25 aa, Homo sapiens
ETYGEMADCCAKQEPERNECFLQHK

SEQ ID NO: 5 - protein, 18 aa, Homo sapiens
FPKAEFAEVSKLVTDLTK

SEQ ID NO: 6 - protein, 18 aa, Homo sapiens
TPVSDRVTKCCTESLVNR

SEQ ID NO: 7 - protein, 12 aa, Homo sapiens
YICENQDSISSK

SEQ ID NO: 8 - protein, 8 aa, Homo sapiens
KYLYEIAR

SEQ ID NO: 9 - protein, 16 aa, Homo sapiens
RHPYFYAPELLFFAKR

SEQ ID NO: 10 - protein, 21 aa, Homo sapiens
AACLLPKLDELRDEGKASSAK

SEQ ID NO: 11 - protein, 15 aa, Homo sapiens
LSQRFPKAEFAEVSK

SEQ ID NO: 12 - protein, 12 aa, Homo sapiens
RHPDYSVVLLLR

SEQ ID NO: 13 - protein, 10 aa, Homo sapiens
ECCEKPLLEK

SEQ ID NO: 14 - protein, 8 aa, Homo sapiens
LCTVATLR

SEQ ID NO: 15 - protein, 7 aa, Homo sapiens
HKPKATK

SEQ ID NO: 16 - protein, 4 aa, Homo sapiens
DAHK

SEQ ID NO: 17 - protein, 4 aa, Homo sapiens
HKPK

SEQ ID NO: 18 - protein, 4 aa, Homo sapiens
YTKK

SEQ ID NO: 19 - protein, 5 aa, Homo sapiens
HPEAK

SEQ ID NO: 20 - protein, 6 aa, Homo sapiens
SEVAHR

SEQ ID NO: 21 - protein, 7 aa, Homo sapiens
ASSAKQR

SEQ ID NO: 22 - protein, 8 aa, Homo sapiens
LKCASLQK

SEQ ID NO: 23 - protein, 9 aa, Homo sapiens
SLHTLFGDK

SEQ ID NO: 24 - protein, 10 aa, Homo sapiens
ECCEKPLLEK

SEQ ID NO: 25 - protein, 22 aa, Homo sapiens
TCVADESAENCDKSLHTLFGDK

SEQ ID NO: 26 - protein, 9 aa, Homo sapiens
AFKAWAVAR

SEQ ID NO: 27 - protein, 13 aa, Homo sapiens
QTALVELVKHKPK

SEQ ID NO: 28 - protein, 29 aa, Homo sapiens
DLGEENFKALVLIAFAQYLQQCPEEDHVK

(19) United States
(12) Patent Application Publication
(10) Pub. No.: US 2008/0199882 A1
(43) Pub. Date: Aug. 21, 2008

(54) METHOD FOR DETECTION OF BIOMARKERS FOR EXPOSURE TO STACHYBOTRYS

(75) Inventors: Dorr G. Dearborn, Mayfield Village, OH (US); Iwona Yike, Chardon, OH (US); Anne M. Distler, Westlake, OH (US); Charles Hoppel, Cleveland Heights, OH (US)

Correspondence Address: HAHN LOESER & PARKS, LLP, One GOJO Plaza, Suite 300, AKRON, OH, 44311-1076, US

(73) Assignee: CASE WESTERN RESERVE UNIVERSITY, Cleveland, OH (US)

(21) Appl. No.: 11/935,384

(22) Filed: Nov. 5, 2007

Related U.S. Application Data
(60) Provisional application No. 60/864,215, filed on Nov. 3, 2006.

(51) Int. Cl. G01N 33/53

(52) U.S. Cl. 435/7.1; 436/501

(57) ABSTRACT

A method of determining exposure of an individual to a macrocyclic trichothecene comprises isolating a sample of at least a portion of a naturally occurring protein from an individual, and detecting a reaction of the sample with a macrocyclic trichothecene. The macrocyclic trichothecene may be a product of Stachybotrys, such as satratoxin G. The sample may be a blood protein or a metabolic product of a satratoxin adduct found in urine. The interaction of the protein and the macrocyclic trichothecene may be detected by any one or more of a number of methods. The sample may be a proteinase product of a naturally occurring protein. A kit for determining the exposure of an individual to a macrocyclic trichothecene comprises an antibody that reacts to a macrocyclic trichothecene covalently bound to an amino acid derived from a naturally occurring protein.

### CROSS REFERENCE TO RELATED APPLICATIONS

This application claims benefit of pending U.S. Provisional Application No. 60/864,215 filed on Nov. 3, 2006.

### STATEMENT REGARDING FEDERALLY SPONSORED RESEARCH OR DEVELOPMENT

This invention was made with government support under NIH grant T23 DK007319, NIEHS grant ES014653 and US EPA co-op agreement CR-82794201. The U.S. government may have certain rights to the invention.

### BACKGROUND OF THE INVENTION

The toxigenic fungus Stachybotrys chartarum is one of several environmental fungi which produce very potent compounds toxic to humans and animals. Although S. chartarum produces several classes of mycotoxins, of greatest concerns are macrocyclic trichothecenes, the most potent members of a large family of trichothecenes. These mycotoxins bind to a single binding site on eukaryotic ribosomes and directly inhibit either initiation, elongation, or termination of protein synthesis depending on which trichothecene is bound. Early investigations of trichothecenes were supported by the Department of Defense since they are chemical warfare agents (T-2 toxin was thought to be the active component of “yellow rain” which the US accused the USSR of using in Viet Nam and Afghanistan) and Iraq had been known to have a stockpile of trichothecenes.

Other toxins produced by S. chartarum include: phenylspirodrimanes, inhibitors of complement especially C₅; cyclosporin, an immune suppressant targeting T-lymphocytes; and stachybocins, a class of mycotoxins that are endothelin receptor antagonists. S. chartarum does not appear to produce an infection, rather, inhalation produces a mycotoxicosis. While spore germination in the lungs of immature rat pups (4 d old) has been reported, there was no subsequent infection established.

The organic dust syndrome from occupational exposures of farm workers to S. chartarum is well described and includes nasal and tracheal bleeding, skin irritation, and alterations in white blood cell counts. High dose exposure to the simple trichothecene deoxynivalenol (DON, vomitoxin) causes emesis and myocardial and gastrointestinal hemorrhages; lower exposures cause damage to the immune system, affect the appetite center of the brain and alter neurotransmitter levels. Primarily because of the immunotoxicity of deoxynivalenol, there are WHO guidelines on the amount of this mycotoxin allowed in wheat.

Concern about S. chartarum in indoor environments surfaced in the mid 1980's. Case reports in both residential and the non-industrial workplace suggested that chronic indoor exposures could result in a variety of debilitating respiratory and non-respiratory symptoms perhaps including an effect on immune function. Recent reviews on toxic mold-related health effects raise both concern and controversy.

In 1994, an outbreak of acute pulmonary hemorrhage in young infants in Cleveland was recognized. The initial case-control investigation led by the Centers for Disease Control (CDC) found an association of this often fatal disorder with the presence of S. chartarum in the water damaged homes of these infants. While subsequent review by the CDC of the initial field investigation of 10 infant cases and 30 controls has questioned the strength of the association, an additional 30 cases were subsequently cared for with the continued observation that almost 90% of these infants come from homes with documented S. chartarum. Others have also seen pulmonary hemorrhage in infants with toxigenic mold exposure and in a 7 year old. The non-pulmonary manifestations are similar to those described in animals exposed to S. chartarum and are consistent with the immune suppressive, neurotoxic, and hemolytic effects of the trichothecenes and/or accompanying mycotoxins. The CDC is currently conducting incidence surveillance nation-wide for this disorder. Informal surveillance identified over 100 other cases of acute infant pulmonary hemorrhage that had been diagnosed across the country between 1993 and 1997.

Stachybotrys requires water-saturated cellulose to grow. Finding it in an indoor environment is an indication of significant water intrusion and damage. While still wet, the spores are sticky, but when dry they are readily aerosolized and stream orient to give an effective aerodynamic diameter of 5.2 μm allowing inhalation out to the distal airways. Even smaller spore and hyphal fragments also contain Stachybotrys mycotoxins. Stachybotrys has been thought to be an infrequent component in residential mold growths; however, using a species-specific genomic probe and quantitative PCR, it was recently found in 71% of moldy homes in Cleveland (while only seen in 3.5% of parallel dust aliquots from these 140 homes using more commonly used culture methods). This reflects its loss of viability upon drying, conditions which still maintain the toxicity and antigenicity of the fungal spores. Of general concern, a survey of the homes of 12,842 children from 24 North American communities and reported the prevalence of mold growth to be 36%.

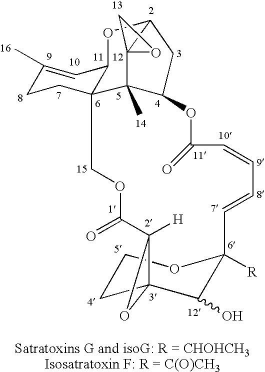

The five primary macrocyclic trichothecenes S. chartarum is capable of producing include, satratoxin G, satratoxin H, isosatratoxin F, roridin E, and verrucarin J. The first and third of this series contain a second epoxide. In a survey of 122 Stachybotrys isolates from damp buildings in Europe and the USA two chemotypes were found, one which always produced the first three macrocyclic trichothecenes and occasionally the last two, and another chemotype which produced atranones and simple trichothecenes (e.g. trichodermin) but never any of the macrocyclic trichothecenes. None of the 122 isolates was found to produce both macrocyclic trichothecenes and atranones. These two chemotypes can grow adjacent to one another in the same building. Ten of 22 isolates from one city produced macrocyclic trichothecenes. While animal models of pulmonary injury demonstrate that macrocyclic trichothecenes are not the only source of lung damage, instilled spores which contain these mycotoxins are significantly more deleterious. The S. chartarum macrocyclic trichothecenes are at least 100 times more potent than the simple trichothecene DON.

As potent protein synthesis inhibitors, trichothecenes cause severe damage to actively dividing cells and have been investigated as potential anti-neoplastic agents in humans; however, the efficacy/toxicity ratio was too small for them to be value. They can be both immunosuppressive and immunostimulatory depending on the dose and exposure regimen. Immunosuppression from trichothecene doses lower than those causing acute cell death promote rapid onset of leukocyte apoptosis in parallel to the effect of cycloheximide. Immunostimulation at even lower doses produces cytokine (e.g. IL-2, IL-6) superinduction which stems from a differential sensitivity to protein synthesis inhibition of cellular regulatory elements, e.g. decreasing IkBα synthesis releasing transcription factor NF-kβ. Similar dysregulation has been observed to occur through alteration of the Jun and Fos family proteins' modulation of the activity of AP-1 transcription factor. Their cytotoxicity and apoptosis induction appear to involve mitogen-activated protein kinases (MAPKs), ERK, p38MAPK and SAPK/JNK.

The association of Stachybotrys inhalation exposure with alveolar hemorrhage in the human infant led to studies of Stachybotrys spore instillation in infant rats. This acute exposure model has documented the importance of the macrocyclic trichothecenes in the lung injury while noting that spore proteinases and a hemolysin are also pathogenic agents. The millimolar concentration of satratoxin G in the spores is five orders of magnitude greater than the EC50 for protein synthesis inhibition.

Five different candidate methods to detect S. chartarum exposure have been previously investigated: 1) luciferase translation toxicity assay, 2) polyclonal antibodies against stachylysin, a hemolysin from S. chartarum, 3) a monoclonal antibody developed at NIOSH against a species-specific antigen from the spores of S. chartarum, 4) quantitative PCR using species-specific probes for environmental fungi including S. chartarum, and 5) polyclonal antibodies against satratoxin G.

The luciferase translation toxicity assay is a sensitive protein synthesis inhibition assay for trichothecenes using luciferase translation which works well with environmental air filters. However, attempts to analyze blood and tissue samples have not been successful because of the high background to signal ratio due to inherent interference from proteinases and nucleases. Attempts to use this with environmental dust samples were also unsuccessful because of high background even when hydoxyapatite columns were used to clean-up the dust extracts.

A novel hemolysin, stachylysin, is produced by S. chartarum. Although purification of stachylysin has been complicated by apparent aggregation, at least 3 proteolytic bands in a partially purified preparation have been detected by electrophoretic zymograms. This preparation was used to raise polyclonal antibodies in rabbits, which were used to localize fungal proteins in spores and mycelia and in animal lungs following instillation of S. chartarum spores. The same affinity purified, polyclonal antibodies were used to develop a competitive ELISA assay with a sensitivity of 2 ppb. This assay was used to quantify stachylysin in the conidia of 91 common indoor fungi, finding that only five other species contained stachylysin and at levels at least 2000 fold lower than S. chartarum, suggesting high species specificity of the assay. Stachylysin was detected in both serum and bronchoalveolar lavage fluid (BALf) from animals following spore instillation. While no stachylysin could be detected in the sera or BALf of infants with pulmonary hemorrhage and documented exposure to S. chartarum, it has been measured in their tracheal secretions. These antibodies have been used in immunocytologic detection of stachylysin in alveolar macrophages of rats exposed to S. chartarum; however, both the sensitivity of detection (˜1000 sp/gm of body wt) and its specificity (false positive results) remain problematic. Thus, caution is necessary in using these antibodies to detect Stachybotrys exposure in light of the immunoreactivity against multiple proteins and practical questions of specificity.

Recently, a monoclonal antibody against a spore-specific antigen has been developed at NIOSH. This is a highly species-specific, high affinity antibody that reacts only with an 18 kD spore protein of S. chartarum. Unsuccessful attempts to detect this protein in BALf and sera from rats exposed to high doses of the spores of S. chartarum in addition to low sensitivity of Western blots (50 μg fungal proteins/lane) and ELISA (5000 spores/ml) indicate that this antigen is not very abundant in the spores and does not appear to be suitable for high sensitivity assays.

The quantitative detection of S. chartarum and other environmental fungi using real time PCR (TaqMan™ fluorogenic probe system) has been developed. This approach has been used in environmental dust and air sample analysis for S. chartarum and other molds. The method measures 2 to 2×10⁵ cells and has much higher sensitivity compared to the conventional culturing approach. PCR assays for 82 species were used to identify and quantify fungal concentrations in dust from homes of infants who developed pulmonary hemorrhage compared to dust from reference homes without visible mold. The ratios of the geometric means of pulmonary hemorrhage houses (PHH) to reference homes (RH) were >1 for 26 fungal species and <1 for 10 other species. Probit analysis of the sum of the logs of the concentrations of these two groups resulted in a 95% probability range for separating PHH from RH homes.

While QPCR analysis is currently the most sensitive and species-specific method of detection, the levels of fungal DNA appear to be very low in biological samples from mold exposed subjects. Without infectious amplification, it is not likely that this assay could be successfully applied to blood samples. Tracheal secretions appear to be more suitable but require more invasive collection techniques e.g. bronchoscopy, and are likely very time-limited relative to exposure. As discussed above, there are at least two chemotypes of Stachybotrys with only one producing the macrocyclic trichothecenes. Thus, finding Stachybotrys DNA in environmental or biological samples does not equate to finding the more deleterious chemotype.

Polyclonal rabbit antibodies raised against a satratoxin G-bovine serum albumin conjugate have been used for body fluid analyses and both histo- and cyto-immunochemical localization. This antibody is highly specific for satratoxin G and other macrocyclic trichothecenes but not for simple trichothecenes. However, only about 0.13% of satratoxin G was recovered in the blood of rat pups exposed to S. chartarum spores (equivalent satratoxin G dose of 0.4 μgm/gm body wt) when the animals were sacrificed immediately following spore instillation. No free toxin in the blood could subsequently be detected by ELISA indicating a very rapid clearance of free toxin. Also, this underlines the lack of adequate sensitivity of the ELISA to detect free satratoxin G in body fluids. This method was also not adequately sensitive to detect satratoxin G in house dust samples with QPCR evidence of S. chartarum.

We have used these antibodies to localize satratoxin in the spores of S. chartarum and in vivo in the lungs of mice exposed intra-tracheally. Although labeling was readily detected in alveolar macrophages, subsequent studies at lower doses indicate that immunocytology also lacks sufficient sensitivity to detect satratroxin in the cells of body fluids.

A satratoxin G-ELISA (described below in Materials and Methods) has been used to detect the toxin in organic solvent extracts of the sera of patients exposed to molds through indoor air inhalation. The removal of proteins prior to analysis precludes the detection of albumin-toxin adducts, although some amino acid and small peptide toxin adducts may be present in those extracts. The majority of their positive clinical samples contained immuno-reactive toxin very close to the limit of detection. Mass spectrometry analysis detected possible toxin breakdown products with spectral properties similar to those of macrocyclic trichothecenes, but the exact nature and origin of the detected substance is not known. For this approach to be quantitatively useful in assessing S. chartarum exposure, the nature and kinetics of satratoxin G metabolism in humans has to be better understood. In addition, the animal experiments described herein showing rapid loss of detectable free toxin from the blood following inhalation-type exposure suggest this analytical approach to be of limited value.

Therefore, there are currently no reliable means to document recent exposure to Stachybotrys or its toxins. An acute quantitative biomarker would be very valuable in the differential diagnoses of infants presenting with pulmonary hemorrhage, e.g. helping to distinguish the stachybotryotoxicoses from other causes. Along with PCR documentation of the organism in tracheal secretions (as seen in four recent cases), acute biomarker evidence is crucial in establishing a causal linkage of S. chartarum to pulmonary hemorrhage in infants; this is currently based only on circumstantial evidence.

The lack of proper biomarkers has greatly hampered investigations of the human health effects of the toxigenic fungus, Stachybotrys. Attempts to relate inhalation exposure to pulmonary, immune and neurological toxicities have had only circumstantial evidence supporting the apparent exposure. Epidemiological and pathophysiological studies would be greatly facilitated by the development of biomarkers that can be quantitatively related to the dose and timing of the exposure.

Conclusive evidence regarding the etiology of the infant pulmonary hemorrhage disorder awaits the development of proper biomarkers. Whether it is infants, older children or adults, it is very apparent that the controversy surrounding the significance of inhalation exposure to ‘toxic mold’ will continue until quantitative, dosimeter biomarkers are available and used in proper epidemiologic studies. Finding Stachybotrys in a patient's home or work environment remains circumstantial evidence without a biological marker documenting the extent and timing of the exposure. With as high as 36% of North American homes having mold growth and a finding of 71% moldy homes in Cleveland containing Stachybotrys, the extent of the health impact is yet to be recognized.

Therefore there is a need for a method of detecting exposure of an individual to macrocyclic trichothecenes.

### SUMMARY OF INVENTION

The present invention takes advantage of the fact that satratoxin G, a macrocyclic trichothecene from Stachybotrys with two epoxides, forms stable adducts with naturally occurring proteins such as human serum albumin. This offers the potential for quantitative biomarker ‘dosimeters’ in the form of protein adducts and related urinary metabolites. It is, therefore, an aspect of the present invention to provide a method of detecting exposure of an individual to macrocyclic trichothecenes. The method may be utilized for veterinary applications.

It is another aspect of the present invention to provide a kit for determining the exposure of an individual to a macrocyclic trichothecene.

In general, the present invention provides a method of determining exposure of an individual to a macrocyclic trichothecene, such as those produced by toxigenic Stachybotrys species like S. chartarum. The method comprises isolating a sample of at least a portion of a naturally occurring protein from an individual, reacting the sample of at least a portion of naturally occurring protein with an antibody against a macrocyclic trichothecene amino acid adduct, and detecting a reaction of the antibody with the sample of at least a portion of a naturally occurring protein. The macrocyclic trichothecene may be a product of Stachybotrys, such as satratoxin G. The sample of at least a portion of a naturally occurring protein may be albumin, such as serum albumin, or a metabolic product of a satratoxin adduct found in urine. The covalent adduct of the macrocylic trichothecene toxin with the naturally occurring protein may be detected by any one of a number of methods, including mass spectrometry, Western blot or Enzyme-linked immunosorbent assay (ELISA). The sample of at least a portion of a naturally occurring protein may be a proteinase product of a naturally occurring protein. The portion of a naturally occurring protein may be a polypeptide that contains at least one amino acid capable of acting as a satratoxin adduct formation site. Such amino acids include lysine, histidine and cysteine.

A kit for determining the exposure of an individual to a macrocyclic trichothecene comprises an antibody against a macrocyclic trichothecene. The antibody is capable of reacting to an adduct of at least a portion of a naturally occurring protein and the macrocyclic trichothecene.

### BRIEF DESCRIPTION OF THE DRAWINGS

FIG. 1 provides the chemical structure of satratoxin G.

FIG. 2 provides an outline of the experiments and analyses reported herein.

FIG. 3 is a graph showing satratoxin G levels over time in the blood of infant rats exposed to the spores of S. chartarum.

FIG. 4 shows the sodium dodecyl sulfate-polyacrylamide gel electrophoresis (SDS-PAGE) and Western blots of human serum albumin incubated (37° C., 20 h, phosphate buffered saline) with different concentrations of satratoxin G (3 μg protein/lane). Western blots were developed with affinity-purified anti-satratoxin G antibody.

FIG. 5A is a portion of the MALDI-TOF mass spectrum of N-α-acetyl-L-lysine incubated (37° C., 20 h, phosphate buffered saline) with equimolar concentration of satratoxin G. A peak is detected at m/z 716.8.

FIG. 5B is an ESI-MS/MS spectrum of the ions at m/z 716.8.

FIG. 6A is an ESI-MS/MS spectrum of tryptic peptides (in the triply charged state) of the ions at m/z 528.33 from rHSA.

FIG. 6B is an ESI-MS/MS spectrum of tryptic peptides (in the triply charged state) of the ions at m/z 474.64 from rHSA.

FIG. 6C is an ESI-MS/MS spectrum of tryptic peptides (in the triply charged state) of the ions at m/z 469.17 from rHSA.

FIG. 7A is a MALDI-TOF spectra of amino acid-satratoxin G adducts in pronase digests of rHSA.

FIG. 7B is a MALDI-TOF spectra of amino acid-satratoxin G adducts in pronase digests of serum albumin from S. chartarum exposed and control patients.

FIG. 8 is a MALDI-TOF spectra of cysteinyl, lysyl and histidinyl satratoxin G adducts from serum of rats exposed to the spores of S. chartarum.

FIG. 9 is a MALDI-TOF spectra for satratoxin G adducts from serum of an unexposed cat (A) and two cats (B and C) exposed to S. chartarum.

### PREFERRED EMBODIMENT FOR CARRYING OUT THE INVENTION

The present invention is directed toward a method and a kit for determining exposure of an individual to macrocyclic trichothecenes, such as satratoxin G. The following examples should not be viewed as limiting the scope of the invention. The claims will serve to define the inventions. It is envisioned that exposure to macrocyclic trichothecenes other than satratoxin G, such as satratoxin H, isosatratoxin F, roridin E, and verrucarin J, may be detected in the present invention similar to the methods described in the following examples. Additionally, it is also envisioned that naturally occurring proteins other than albumin may also be used to detect the formation of adducts of macrocyclic trichothecenes.

Mycotoxins containing epoxides form adducts with host proteins as shown when satratoxin G was incubated with serum albumin followed by electrophoresis and Western blotting with anti-satratoxin G antibodies. The protein bands did show immunoreactivity evidence of possible satratoxin G adducts with these proteins as described herein. To demonstrate that satratoxin G can form adducts with a serum albumin in vitro, several mass spectrometry (MS) techniques were used. The intact protein (recombinant human serum albumin; rHSA; GTC Biotherapeutics Inc, Spencer, Mass.) was analyzed with and without satratoxin G treatment. When the untreated rHSA was analyzed using an electrospray ionization (ESI) source coupled to a quadupole time-of-flight (TOF) instrument, several protein species ranging from 66,000-67,000 Da were detected. When analyzing the treated serum albumin, protein species were detected ranging from 66,000-71,000 Da. Based on the resolution and mass accuracy of this mass spectrometer, it can be estimated that up to 10 satratoxin G molecules can bind to the protein. Using matrix-assisted laser desorption ionization (MALDI) TOF MS, these results were confirmed.

While analysis of the intact protein can determine the number of toxin molecules bound to the protein, the exact site of the modification cannot be identified. In order to locate the sites of satratoxin G adduction, rHSA was digested with trypsin and the resulting peptides analyzed using mass spectrometry. Using MALDI-TOF MS, the molecular weights of the tryptic peptides were determined. From these masses, peptide mass fingerprinting (PMF) was performed to determine the identity of the peptides by matching the experimental molecular weights with the theoretical values from the protein sequence. When analyzing the tryptic digests of the rHSA, 40-64% sequence coverage of the protein was seen using MALDI-TOF MS and PMF. There were several ions detected in the MS experiment that did not correspond to those predicted tryptic peptides. These ions were consistent by mass and sequence as peptides of HSA with one satratoxin G molecule adducted to the peptide. The peptide-satratoxin G adducts detected in the MALDI-TOF MS experiment are listed in Table 1 below.

During an MS/MS experiment, an ion is selected, fragmented, and the fragment ions detected. When analyzing peptides, the ions fragment along the peptide bond allowing for facile structure elucidation and sequence determination. For this reason, the tryptic digests were also analyzed using ESI coupled to a quadrupole ion trap, an instrument with MS/MS capabilities. When the tryptic digests of the modified rHSA were analyzed using ESI-MS/MS, 40% of the protein was sequenced. Tandem MS was used to identify peptide sequences and position of modified residues. For example, a peptide was identified as ⁷⁴LC_{SG}TVATLR⁸¹ where there was a mass shift of 544 Da on the cysteinyl residue indicating the presence of satratoxin. Another peptide was identified as ¹³⁷K_{SG}YLYEIAR¹⁴⁴ with a satratoxin adduct of +528 on the lysyl residue.

In seeking sensitive and informative biomarkers, it is advantageous to use the most abundant source and those with relatively long biological half-lives. All of the cells and proteins within whole blood have extensive inhalation exposure in the lung with minimal barriers and metabolism (in contrast to gastrointestinal exposure). The two most abundant proteins in whole blood are hemoglobin (12-14 mg/dL) and serum albumin (3-4.5 mg/dL) with about 28 d and 20 d half-lives respectively. Given this, it is envisioned that both proteins are practical sources of satratoxin adducts thereby providing differential time profiles for shorter and longer exposure markers. The extensive experience in diabetes with glycohemoglobin (Hgb-A1c) provides an important background for such considerations.

Satratoxin G adduct formation may be determined by isolating protein components of whole blood such as albumin or hemoglobin. Simple centrifugation (600 g×10 min) and discarding of the white cell buffy coat will give primary separation of these two major blood proteins.

Excess calcium may be added to the plasma fraction for coagulation and centrifugation used to harvest the serum. The albumin-SG adduct may be detected by immunoaffinity detection such as Western blot analysis and/or LC/MS identification of the satratoxin-amino acid adduct following exhaustive proteolysis with pronase and aminopeptidase. Erythrocytes may be subjected to freeze-thawing and dilution in hypotonic EDTA for lysis, the ghosts removed by centrifugation and hemoglobin acetone precipitated and extracted. The dry globin (e.g. 1-2 mg) may be reconstituted in proteolysis buffer and exhaustively digested as above with pronase and aminopeptidase. The digest may be applied onto a polyclonal antibody affinity column, the column washed with buffer and the adducts eluted with formic acid.

Urine is another convenient source of biomarkers, one that offers a more acute exposure documentation. However, with urine there is the added complexities of toxin and toxin-adduct metabolic products. While there have been no mammalian metabolic studies of macrocyclic trichothecenes, it is envisioned that metabolic products of satratoxin may be readily projected and thereby potentially could be detected in the urine.

### Materials and Methods

### Animal Experiments:

Animals: Sprague-Dawley rats were obtained from Charles River (Wilmington, Mass.). All animals were housed in microisolators in the Case Western Reserve University (CWRU) animal facility and fed the standard diet of Teklad 8664 (Harlan) and water ad libitum. The animal research protocol has been approved by the Case Western Reserve University Institutional Animal Care and Use Committee. The animals were treated humanely and with regard for the alleviation of suffering.

Intratracheal instillation of fungal spores: Seven day old rat pups weighing around 10 g were used for measurements of satratoxin G in blood. Male and female rats weighing 100 g were used to search for satratoxin G albumin-adducts. In both cases, the animals were anesthetized with isoflurane (Baxter, Deerfield, Ill.). A transverse skin incision was made and the trachea exposed by blunt dissection. S. chartarum spores (isolate JS58-17; 4.0×10⁵ spores per gram body weight in satratoxin G measuring experiments and 0.5×10⁵ spores per gram body weight in albumin-adduct detection experiments) suspended in 50 μl of PBS containing 0.1% Tween-20 were injected directly into the trachea using a 24 G angio-catheter attached to a sterile Hamilton syringe. The incision was closed and treated with New Skin liquid bandage (Medtech Laboratories Inc., Jackson. Wyo.) to facilitate healing.

Collection of blood: The animals were exsanguinated through the right ventricle under isoflurane anesthesia at indicated times after spore instillation and the blood samples were combined using three animals per time point. For satratoxin G measurements, the blood was immediately extracted with ethanol (see below) while for albumin-adduct detection, the blood was centrifuged at 1000×g for 10 minutes and the serum aspirated and stored at −20° C.

### Human Blood Samples:

Blood samples were obtained from three adult patients with documented exposure to S. chartarum who were evaluated at the Environmental Health Clinic, Swetland Center for Environmental Health, Case Western Reserve University. The use of human samples was reviewed and approved by the Institutional Review Board of the University Hospitals of Cleveland and all recruited patients gave informed consent. The sample from patient #1 was taken approximately 2 months after the completion of mold remediation in her home. Previous air sampling in her living room had measured 4,700 S. chartarum spores/M³ (Air-O-Cell; US Micro-Solutions, Inc, Greensburg, Pa.). Blood samples from patients #2 and #3 were obtained while they were still living in their home where S. chartarum was found in their bedroom carpeting (1,030 S. chartarum spore equivalents/gm; quantitative Polymerase Chain Reaction, P & K Microbiology Services, Cherry Hill, N.J.). Control blood samples were received from the investigators. The blood was centrifuged and the serum stored at −20° C.

### Anti-Satratoxin G Antibodies and Satratoxin G ELISA.

Polyclonal antibodies against satratoxin G were generously donated by Dr. James Pestka, Michigan State University. The IgG fraction, obtained by ammonium sulfate precipitation from the sera of rabbits immunized with the conjugates of bovine serum albumin and satratoxin G (Chung et al. 2003), was purified by passing through an affinity column of bovine serum albumin (Sigma-Aldrich, St. Louis, Mo.) conjugated to Amino Link® Sepharose (Pierce, Rockford, Ill.) to remove antibodies specific for bovine albumin epitopes.

Satratoxin G was measured by ELISA (detection limit=0.1 ng/ml) in 1:1 whole blood ethanol extracts according to the method of Chung et al. (2003) using anti-satratoxin G antibody and horseradish peroxidase-satratoxin G conjugates provided by Dr. James Pestka from Michigan State University.

### Formation of Albumin-Satratoxin G Adducts In Vitro:

Human serum albumin isolated from human blood (Sigma-Aldrich, St. Louis, Mo.) and/or recombinant serum albumin (rHSA, GTC Biotherapeutics, Spencer, Mass.) was dissolved in phosphate buffered saline at 10 μM concentration and incubated for 20 h at 37° C. with 0, 1, 2, 5, 10 and 20 molar excess of satratoxin G (gift from Dr. Bruce Jarvis, University of Maryland). The samples were then subjected to SDS-PAGE followed by Western blots or dialyzed and analyzed by mass spectrometry as described below. A molar ratio of 1:10 of albumin to satratoxin G was used for MS studies.

N-α-acetyl-L-lysine (Sigma-Aldrich, St. Louis, Mo.) was dissolved in phosphate buffered saline at 10 μM concentration and incubated for 20 h at 37° C. with an equimolar concentration of satratoxin G.

### Electrophoresis and Western Blotting:

SDS-PAGE was performed using 12% pre-cast Criterion gels (Bio-Rad, Hercules, Calif.). The proteins were transferred to nitrocellulose and Western blots were developed using 1:1000 diluted (affinity purified as above) anti-satratoxin G antibody. Alkaline-phosphatase anti-rabbit IgG (Sigma-Aldrich, St. Louis, Mo.) was used as a secondary antibody and the protein bands were visualized with alkaline phosphatase substrate kit from Bio-Rad (Hercules, Calif.).

### Preparation of Samples for MS Analysis:

Tryptic digestion of rHSA and human serum: rHSA was incubated with (1:10 molar ratio of to SG) and without satratoxin G as described above and dialyzed against 20 mM ammonium bicarbonate pH 7.8. Dialyzed protein was incubated overnight at 37° C. with sequencing grade trypsin (Promega, Madison, Wis.) at 1:100 w/w ratio. Samples of human serum (1-3 mL) were similarly digested with a TCPK trypsin preparation from Sigma-Aldrich (St. Louis, Mo.).

Exhaustive digestion of rHSA and serum: rHSA and rHSA-satratoxin G adducts, dialyzed overnight against 20 mM ammonium bicarbonate pH 7.8, were incubated overnight at 37° C. with pronase (Calbiochem®, EMD Biosciences, San Diego, Calif.) at 1:100 w/w ratio and leucine aminopeptidase (Sigma-Aldrich, St Louis, Mo.) at 1:1000 w/w ratio. Human and rat serum (1-3 mL) were similarly digested except using the leucine aminopeptidase at 1:500 w/w. If the digestion to single amino acids was not complete, carboxypeptidase Y (Sigma-Aldrich, St. Louis, Mo.) was added to reconstituted samples for 4 h and the analysis repeated.

Affinity chromatography: Both trypsin and pronase digested samples were heat-inactivated at 60° C. for 30 minutes, centrifuged at 17000×g for 20 minutes prior to affinity chromatography. The affinity column for isolating peptide and amino acid adducts was prepared by conjugating anti-satratoxin G antibody (see above) to AminoLink® Sepharose. Heat-inactivated tryptic and/or pronase digests from human and rat serum were loaded onto the column and incubated for 1 h at room temperature. The column was extensively washed with phosphate buffered saline followed by 20 mM ammonium bicarbonate pH 7.8. Bound adducts were eluted with 0.02% formic acid and evaporated. The samples were reconstituted in 0.1% formic acid and analyzed by mass spectrometry.

### Mass Spectrometry Instrumentation and Analyses:

Intact protein: Intact rHSA was analyzed using an Applied Biosystems (Framingham, Mass.) Q-STAR XL quadrupole time-of-flight (TOF) mass spectrometer equipped with a nanospray source or a Bruker (Billerica, Mass.) Biflex III TOF mass spectrometer with a matrix-assisted laser desorption ionization (MALDI) source. For intact protein analysis by MALDI-TOF MS, sinapinic acid was used as the matrix. One microliter of the saturated matrix mixture (in a 1:1 acetonitrile: water solution) was spotted on target with one microliter of the protein solution.

Tryptic Peptide Identification: After rHSA digestion with trypsin, the resulting peptides were analyzed using MALDI-TOF MS to determine their molecular weights. For MALDI-TOF MS analysis of the peptides, the matrix, a-cyano-4-hydroxy cinnamic acid, was used. One microliter of the saturated matrix (in a 1:1 acetonitrile: water solutions) was spotted on target with 1 microliter of the analyte. Using the m/z values from the mass spectra, peptide mass fingerprinting (PMF) was performed to determine the identity of the peptides by matching the experimental molecular weights with the theoretical values calculated from the protein sequence.

To further confirm the identity of the peptides and locate the modified amino acids, tandem mass spectrometry (MS/MS) was performed using the ThermoElectron LCQ-Deca XP plus ion trap mass spectrometer with nanospray source. Tryptic digests were diluted in 1% acetic acid and 2 μl of each sample were pressure injected onto a self-packed 10 cm×75 μm ID Phenomenex Jupiter C18 reversed-phase capillary column. The peptides were eluted from the column by an acetonitrile and aqueous 0.05 M acetic acid gradient with a flow rate of approximately 0.25 μL/min at the nanospray tip. The digest was analyzed by acquiring full scan mass spectra followed by MS/MS. The three most abundant ions detected in the full scan mass spectrum were then selected and fragmented to yield the MS/MS spectrum of the peptide. The MS/MS data were analyzed using the ThermoElectron BioWorks 3.1 program. All matching spectra were verified manually.

The pronase digests were analyzed on the MALDI-TOF mass spectrometer using the same matrix and sample preparation detailed above for the tryptic digest analysis. The limit of detection for this analysis of human samples was approximately 10 nanomoles/mL of serum.

### Results

### Detection of Satratoxin G in Blood:

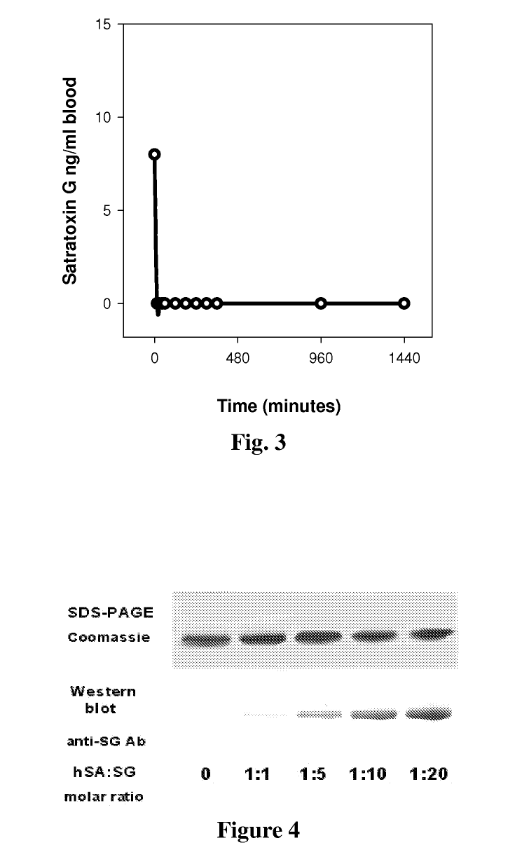

When 7 d old infant rat pups were exposed intra-tracheally to high doses (4.0×10⁵ spores/gm body weight) of highly toxic S. chartarum (isolate JS-58-17), satratoxin G was detected in ethanol extracts of the whole blood by satratoxin G-ELISA only immediately after instillation (FIG. 3). The level of free toxin decreased below the detection limit within the next 15 minutes. No immunoreactive free toxin could be detected in the sera of exposed animals between 1 h-72 h post exposure, demonstrating the difficulty of directly detecting exposure of an individual to macrocyclic trichothecenes with prior methods.

### Satratoxin G Adducts In Vitro:

Detection of Anti-Satratoxin G-Reactive Albumin:

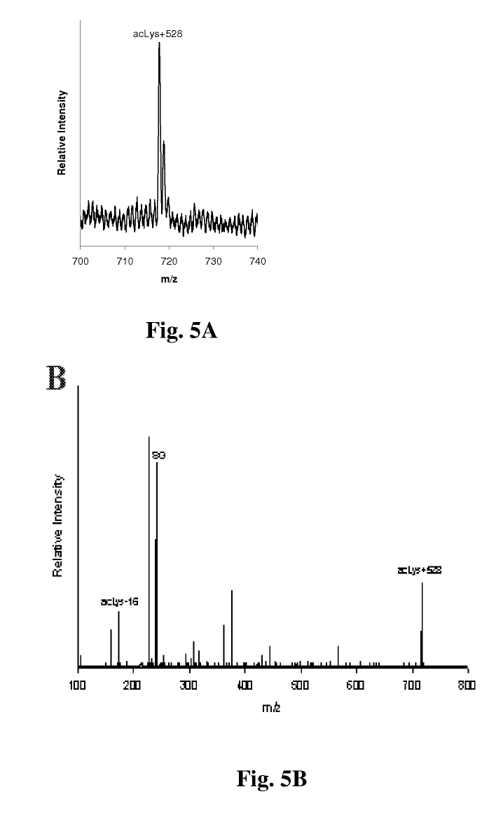

Samples of purified human serum albumin (HSA; Sigma-Aldrich) were incubated with increasing concentrations of satratoxin G (PBS, 37° C., 20 h) and subjected to SDS-PAGE following reduction and boiling of the samples. Western blots using anti-satratoxin G antibody clearly demonstrate the HSA band at ˜67 kDa (FIG. 4) with the intensity of staining increasing with increasing concentrations of satratoxin G. This concentration dependence of the satratoxin G staining which persists through boiling and SDS electrophoresis supports the formation of covalent satratoxin G-albumin adducts. These results were confirmed using the recombinant human serum albumin (rHSA) from GTC Biotherapeutics.

MS Analysis of Intact Protein:

When untreated and satratoxin G-incubated rHSA (20 h, 37° C., PBS, 1:10 protein to toxin ratio) were analyzed using an ESI-q TOF mass spectrometer, a mass shift was seen in the treated rHSA sample. This molecular weight increase was indicative of as many as ten satratoxin molecules bound to the protein. These results were further confirmed using a MALDI-TOF mass spectrometer (data not shown).

MS Analysis of Satratoxin G-N-Acetyl-Lysine Adduct:

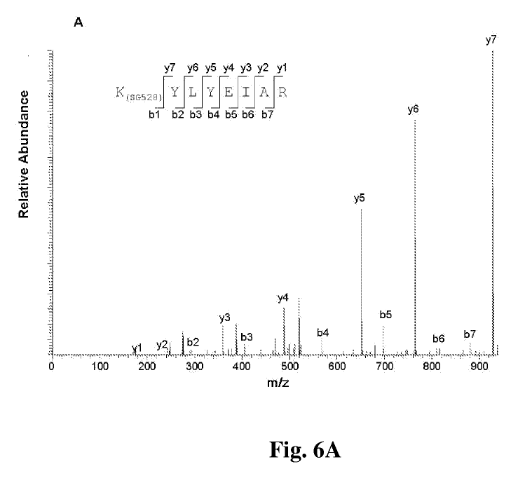

Since the ε-amine of lysyl residues is a likely site of satratoxin G nucleophilic attack, we incubated the toxin with N-α-acetyl-L-lysine (1:1 molar ratio, 37° C., 20 h) and analyzed the resulting satratoxin-lysyl adduct. FIG. 5 shows the spectra from both MALDI-TOF (A) and ESI-MS/MS (B) of the resulting satratoxin-lysyl adduct. The m/z 716 is consistent with the addition of 528, an apparent loss of oxygen when the satratoxin G bound to the amino acid. When the ions at m/z 716 were isolated and fragmented, the MS/MS spectrum contains a peak at 172 representing the N-acetyl lysine as well as a peak representing a fragment of the toxin at m/z 239, a convenient marker for this adduct in tandem MS experiments. This peak at 239 Da is detected in both the mass spectrum of the toxin molecule alone and as a fragment ion in the MS/MS spectrum of the toxin molecule.

Detection of Adducted Tryptic Peptides from rHSA:

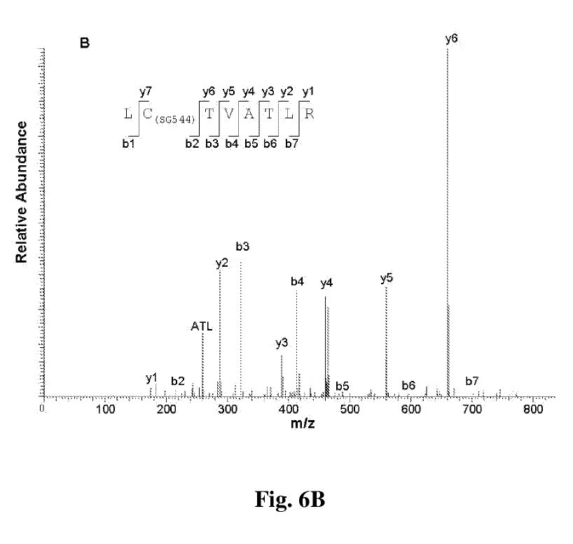

In order to locate the sites of satratoxin G adduction to rHSA, both untreated and treated rHSA were digested with trypsin and analyzed with MALDI-TOF MS using peptide mass fingerprinting to identify the peptides. When analyzing the tryptic peptides of treated rHSA, several ions were detected that were consistent with the predicted mass of peptides bound to one satratoxin G molecule (Table 1). To identify the exact position of toxin molecules on these peptides, the tryptic digests of treated and control rHSA were analyzed further using ESI-MS/MS (Table 1, FIG. 6). Around 60% coverage was obtained for both samples. The ions representing tryptic peptides were in the triply charged state. When the ions at m/z 528 were isolated and fragmented, the resulting MS/MS spectrum identified the peptide as K(SG528)YLYEIAR with a calculated mass of 1582 ({528×3}−2). Another labeled peptide was identified when the ions at m/z 475 were isolated and fragmented. The fragmentation pattern in this MS/MS spectrum is consistent with the peptide, LC(SG544)TVATLR with a calculated mass of 1423 Da ({475×3}−2). Lastly, when the ions at m/z 469 were fragmented, the same peptide was identified, but with a +528 satratoxin G adduct ({469×3}−2=1405 Da). As indicated in Table 1, the sequences of adducted tryptic peptides all contained lysyl, cysteinyl, or histidyl residues (see below), residues which are likely to be susceptible to modification by the toxin epoxide groups.

TABLE 1
Adducted tryptic peptides from rHSA.

| | | | |
|---|---|---|---|
| m/z | Amino Acids | Sequence ^{a} | Adduct |

| | | | |
|---|---|---|---|
| 5739.9 | 277-323 | ECCEKPLLEKSHCIAEVEND | 528 |
| | | EMPADLPSLAADFVESKDVC | |
| | | KNYAEAK | |
| 4566.2 | 214-276 | VHTECCHGDLLECADDRADL | 544 |
| | | AKYICENQDSISSKLK | |
| 4565.2 | 485-519 | RPCFSALEVDETYVPKEFNA | 528 |
| | | ETFTFHADICTLSEK | |
| 3487.5 | 82-106 | ETYGEMADCCAKQEPERNEC | 528 |
| | | FLQHK | |
| 2551.5 | 223-240 | FPKAEFAEVSKLVTDLTK | 528 |
| 2551.5 | 467-484 | TPVSDRVTKCCTESLVNR | 544 |
| 1914.5 | 263-274 | YICENQDSISSK | 528 |
| 1582.6 | 137-144 | KYLYEIAR | 528 |
| 2583.3 | 145-160 | RHPYFYAPELLFFAKR | 528 |
| 2743.4 | 175-195 | AACLLPKLDELRDEGKASSAK | 528 |
| 2264.7 | 219-233 | LSQRFPKAEFAEVSK | 528 |
| 1995.5 | 337-348 | RHPDYSVVLLLR | 528 |
| 1719.2 | 277-286 | ECCEKPLLEK | 528 |
| 1404.5 | 74-81 | LCTVATLR | 528 |
| 1420.5 | | LCTVATLR | 544 |
| 1353.6 | 535-541 | HKPKATK | 544 |
| 1014.2 | 1-4 | DAHK | 544 |
| 1037.4 | 535-538 | HKPK | 528 |
| 2599.3 | 145-160 | RHPYFYAPELLFFAKR | 544 |

^{a} Presumed modification sites of peptides analyzed by MALDI-TOF MS are in bold.

Modification sites identified by ESI-MS/MS are underlined.

Detection of Adducted Amino Acids in Pronase Digests of rHSA:

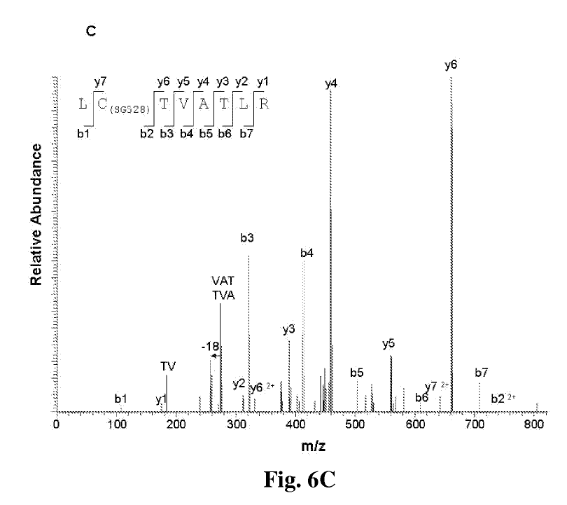

To further characterize the rHSA-satratoxin G reactivity, exhaustive proteolysis of reacted rHSA was performed with pronase and leucine aminopeptidase. The modified amino acids were purified using anti-satratoxin G antibody affinity chromatography and analyzed by MALDI-TOF MS. In the MALDI-TOF mass spectra, several ions were detected corresponding to the amino acids Lys, His, and Cys, each containing one satratoxin G molecule (FIG. 7A). Two different cysteinyl adducts +528 and +544 Da were detected. The amino acid assignments in FIG. 6A were confirmed by ESI-MS/MS (data not shown).

### Satratoxin G-Albumin Adducts In Vivo:

Amino Acid Satratoxin G Adducts in Pronase Digests of Human and Rat Sera:

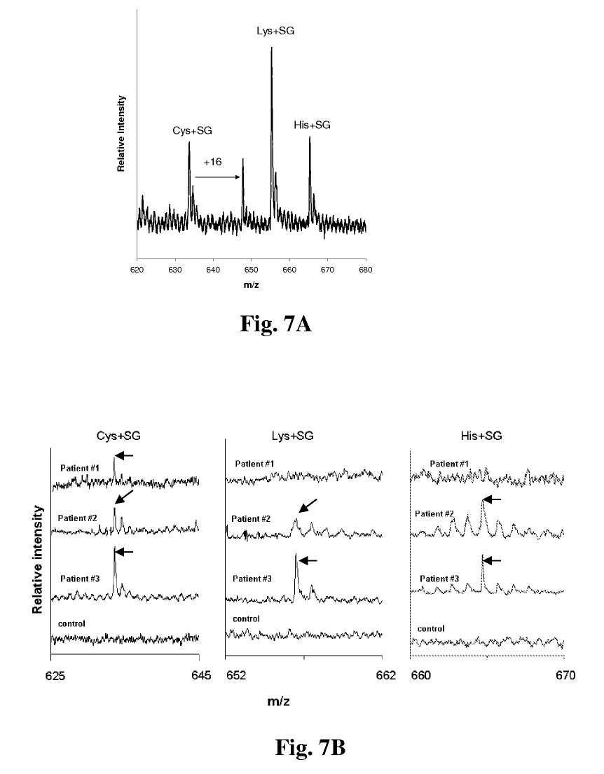

Subsequently, 2 mL serum samples from three patients with documented exposure to S. chartarum (see above) and three control subjects were digested with pronase and leucine aminopeptidase, the adducts affinity purified, and analyzed by MALDI-TOF MS. In FIG. 7B, the three top spectra in each panel were acquired from the samples from exposed patients while the bottom spectra are from one of the control subjects. Patients #2 and #3 had a more recent exposure compared to patient #1. While cysteinyl adducts were detected in all three of the exposed patient samples, lysyl and histidyl adducts were not detected in the patient #1 whose blood sample was collected 2 months after the termination of exposure. No amino acid adducts were detected in the sera from the three control subjects. Similarly, cysteinyl, lysyl and histydyl satratoxin G adducts were detected following exhaustive proteolysis of the sera of rats exposed intra-tracheally to S. chartarum collected six hours after the instillation of fungal spores (FIG. 8). No adducts could be found in parallel samples from control animals.

Adducted Tryptic Peptides from Serum of a Patient Exposed to S. Chartarum:

In order to demonstrate the albumin origin of amino acid adducts detected in samples from human subjects, serum from patient #3 (most recent exposure, all three amino acyl adducts detected) was digested with trypsin and the adducted peptides isolated with anti-satratoxin immunoaffinity chromatography. Analysis by MALDI-TOF MS followed with peptide mass fingerprinting detected eight peptides with the additional mass of 528 Da and four with 544 Da (Table 2). All of the detected tryptic peptides contained at least one of the three amino acyl residues, lysyl, cysteinyl, and histidyl that were identified as probable modification sites in rHSA.

TABLE 2

Tryptic peptides from serum albumin
of a patient exposed to S. chartarum.

| | Amino | | |
|---|---|---|---|
| m/z | Acids | Sequence ^{a} | Adduct |
| 1067.4 | 435-438 | YTKK | 528 |
| 1109.4 | 464-468 | HPEAK | 528 |
| 1226.2 | 29-34 | SEVAHR | 528 |
| 1275.2 | 215-221 | ASSAKQR | 528 |
| 1418.4 | 222-229 | LKCASLQK | 528 |
| 1544.9 | 89-97 | SLHTLFGDK | 528 |
| 1720.3 | 301-310 | ECCEKPLLEK | 528 |
| 2911.1 | 76-97 | TCVADESAENCDKSLHTLFGDK | 528 |
| 1053.4 | 559-562 | HKPK | 544 |
| 1564.0 | 234-242 | AFKAWAVAR | 544 |
| 2035.2 | 550-562 | QTALVELVKHKPK | 544 |
| 3910.1 | 37-65 | DLGEENFKALVLIAFAQYLQQCPEEDHVK | 544 |

^{a} Amino acid residues that may be modified within the peptide are in bold.

With the above results, it should be evident that a macrocylic trichothecene produced by S. chartarum, satratoxin G, forms covalent adducts with human serum albumin and that these adducts can be detected in clinical samples from patients exposed to this fungus.

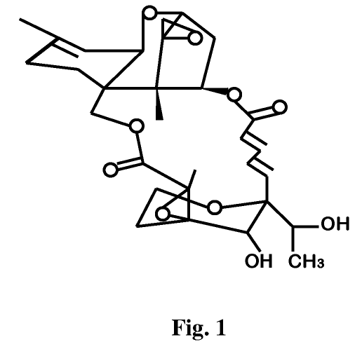

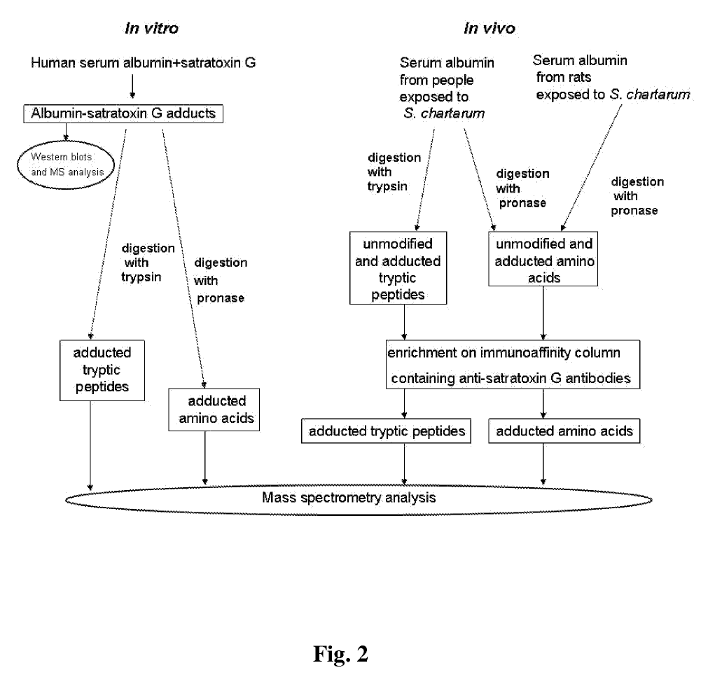

The five primary macrocyclic trichothecenes produced by S. chartarum include satratoxin G (FIG. 1), satratoxin H, isosatratoxin F, roridin E, and verrucarin J. All of them contain an epoxide group that is critical to their toxicity and the first and third of this series contain a second epoxide. These highly reactive groups are likely to be involved in rapid adduct formation with proteins, and with inhalation exposure, this reaction would likely occur with blood proteins in the alveolar capillaries. Therefore, the detection of adducts of any of these compounds with blood proteins or other naturally occurring proteins is also within the scope of the present invention.

The reaction of satratoxin G with tissue and cellular proteins is also likely. Western blots showing the staining of albumin with anti-satratoxin G antibody (FIG. 4) were obtained with reduced and boiled samples further suggesting covalent links between the mycotoxin and protein.

ESI-TOF and MALDI-TOF MS analysis of the intact adducted protein showed that up to ten amino acid residues in the albumin molecule are modified following the incubation of the protein with a 20-fold excess of toxin in vitro. It is also envisioned that the extent of albumin modification in vivo would be dependent upon the level and timing of exposure and is likely to reflect the cumulative nature of chronic exposure. MALDI-TOF MS analysis of exhaustive pronase/aminopeptidase digests of recombinant human serum albumin demonstrated that in addition to lysyl residues two other amino acyl residues, cysteinyl and histidyl, are involved in adduct formation. All of the rHSA derived tryptic peptides with satratoxin G adducts had one of those amino acyl residues within their sequences and Cys₇₅ and Lys₁₃₇ were positively identified as modification sites using ESI MS/MS analysis.

Using an affinity column with anti-satratoxin G antibodies to isolate adducts from proteolytic digests provides a concentrating step which greatly increases the sensitivity of detection. The ability to detect lysine-, cysteine- and histidine-satratoxin G adducts in pronase/aminopeptidase digests of serum from patients exposed to S. chartarum in their homes, in contrast to samples from people without mold exposure, demonstrates the feasibility of a practical biomarker assay. Detection of modified tryptic peptides with albumin sequences in the sample of patient's serum indicates that those amino acyl adducts came from serum albumin, although modification of other serum proteins is likely. In addition, the presence of lysine-, cysteine- and histidine-satratoxin G adducts in the sera from rats exposed intra-tracheally to the spores of a highly toxic isolate of S. chartarum further confirms the biomarker potential of the adducts.

### Veterinary Application

The following example demonstrates the applicability of the method for testing for exposure to Stachybotrys to the veterinary field. Two overtly healthy Himalayan cats from a single household were brought to a veterinary hospital for annual dental cleaning and prophylaxis. The cats were siblings and were housed strictly indoors. Both cats were anesthetized by placing them in an induction chamber and exposing them to 5% isoflurane.

Dental cleaning in cat 1 proceeded without incident until the procedure was almost complete. While polishing the teeth, the dental technician noticed frothy blood in the endotracheal tube and the procedure was halted. Thoracic radiography was performed while the cat recovered from anesthesia and revealed diffuse pulmonary infiltrates consistent with alveolar hemorrhage in the dorsocaudal lung fields (not shown). As a result, the cat was given vitamin K (15 mg, SC) and placed in a heated recovery cage; fluid administration was continued at a maintenance rate. The cat recovered from anesthesia without further incident. No more hemorrhage was seen, but the endotracheal tube was found to be covered with coagulated blood when removed. Following extubation, the cat seemed comfortable in the recovery cage and was clinically eupneic.

The events for cat 2 were similar to those for cat 1, with the exception that laser rhinoplasty was performed prior to initiation of dental cleaning. Again, frothy blood was noticed in the endotracheal tube near the completion of the dental cleaning. As with cat 1, the procedure was halted and the endotracheal tube was suctioned. Again, approximately 3 to 4 mL of bloody fluid was aspirated. Thoracic radiographs revealed diffuse alveolar infiltrates similar to those seen in cat 1.

Vitamin K (15 mg, SC) was administered, and cat 2 was placed in a recovery incubator for monitoring. The cat remained dyspneic, demonstrating open-mouth breathing, and supplemental oxygen bubbled through saline solution was administered.

Cat 1 was discharged later the same day. After returning home, the owners thought that the cat's breathing was labored and brought the cat back to the hospital. On reexamination, the cat appeared to be resting comfortably but moist rales were noticed bilaterally during auscultation over the dorsal lung fields. The cat was hospitalized overnight for monitoring. On the assumption that both cats had a condition similar to exercise-induced pulmonary hemorrhage in horses, for which there is no specific treatment, it was decided to monitor the cats overnight while maintaining them in a quiet, low-stress environment and providing supplemental oxygen as needed.

The following morning (i.e., day 2), both cats were resting comfortably. Cat 2 was removed from the oxygen cage and returned to a standard ward for continued monitoring. In cat 1, however, recheck radiography revealed that the infiltrates had either expanded or migrated from the dorsocaudal lung fields to the cranioventral lung fields (not shown). Later that afternoon, cat 1 became dyspneic and was transferred to an oxygen cage and supportive treatment was initiated. The cat's condition stabilized with treatment, and the cat appeared clinically to be eupneic, but approximately 7 hours later, the cat developed hemoptysis followed by respiratory and cardiac arrest. Attempts at resuscitation were unsuccessful.

The initial treatment for cat 2 was identical to that for cat 1. The cat appeared eupneic and was discharged. Cat 2 was reexamined on day 6. Results of thoracic auscultation were unremarkable, and no infiltrates were seen on thoracic radiographs. On day 9, the owner reported that the cat was doing well and breathing normally but had a poor appetite. The cat's condition appeared stable, and the cat was not febrile.

The owner reported on day 12 that the cat had an improved appetite but had vomited 3 times that morning, with the last vomitus having a tinge of blood. On examination, the cat was lethargic but still eupneic. On day 14, the owners took the cat to another hospital for a second opinion. The following day (day 15), the owners chartered a plane to take the cat for a third opinion at yet another hospital. The cat died later that evening, several hours after the flight.

Gross necropsy findings for cat 1 included severe, diffuse pulmonary edema and hemorrhage in both lungs. Edema fluid was evident in the bronchi and bronchioles, and a small amount of blood-tinged fluid was seen in the trachea and nasal passages. There were no other gross abnormalities and no other evidence of hemorrhage anywhere else in the cat. Histologic examination of lung tissue revealed that the pulmonary architecture had been completely effaced by hemorrhage, fibrin deposition, and large numbers of mixed inflammatory cells. Bacterial culture of a lung specimen yielded a pure growth of Pasteurella spp.

Necropsy of cat 2 revealed bronchopneumonia; bacterial culture of lung specimen yielded β-hemolytic Streptococcus spp and Klebsiella pneumoniae. The pathologist indicated that the lung lesions appeared to be acute (ie, of ≦3 days' duration) and secondary to aspiration. No other evidence of hemorrhage was seen on the gross examination. Microscopically, lung specimens included multifocal areas where bronchioles and surrounding alveoli were partially or completely filled with fibrin, large numbers of bacteria, and abundant cellular debris.

Serum that had been collected and banked from each cat was analyzed for evidence of mold toxin exposure. Samples were analyzed for mycotoxin-serum protein adducts by means of adduct detection as described above. In brief, following exhaustive digestion of serum proteins with pronase, affinity chromatography was performed with polyclonal anti-satratoxin G antibodies that selectively bind macrocyclic trichothecenes and their amino acid and peptide adducts. Adducts were eluted and identified by means of matrix-assisted, laser desorption, time-of-flight mass spectrometry.

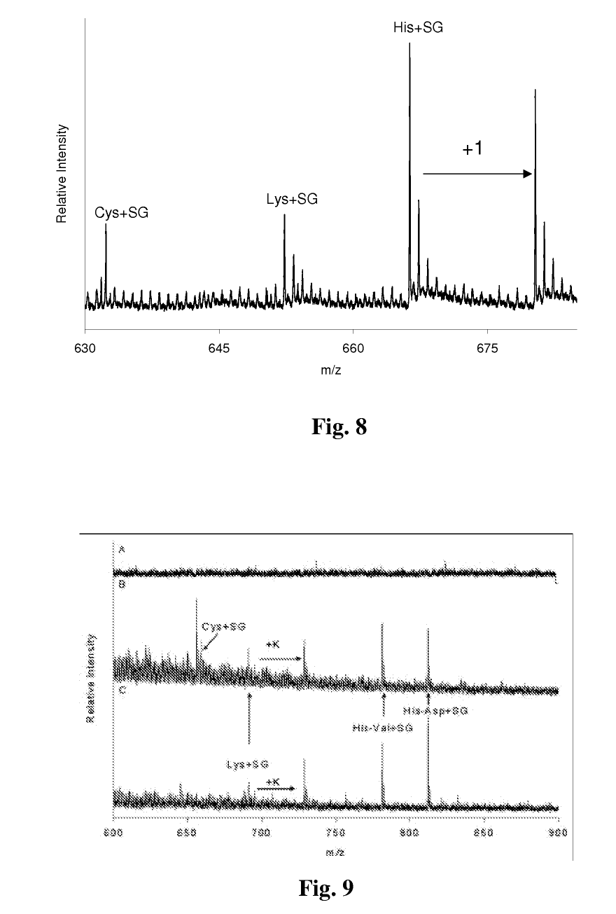

Serum samples from both cats were positive for satratoxin G adducts, biomarkers for the toxin produced by Stachybotrys chartarum. In FIG. 9, MALDI-TOF mass spectrometry for satratoxin G adducts in pronase digests of serum samples from a healthy control cat (A) and from the 2 cats (cat 1 [B] and cat 2 [C]) that developed acute pulmonary hemorrhage during isoflurane anesthesia for routine dental cleaning and prophylaxis are shown. (Cys+SG=Cysteine−satratoxin G adduct. Lys+SG=Lysine−satratoxin G adduct. His−Val+SG=Histidyl valine−satratoxin G dipeptide adduct. His−Asp+SG=Histidyl aspartic acid-satratoxin G dipeptide adduct. +K=Lysine−satratoxin G adduct with bound potassium.) Cysteinyl-satratoxin G and lysyl-satratoxin G adducts were found in the serum of cat 1. Similarly, the adduct of lysine with satratoxin G was detected in the serum of cat 2. Lysine-satratoxin G adducts containing potassium were detected in serum from both cats, along with 2 dipeptidyl-satratoxin G adducts. Serum samples from 6 healthy blood-donor cats living in a mold-free environment in Denver, Colo., were also tested as controls. None of the 6 control cats had satratoxin G adducts in their serum.

The 2 cats in the study had acute pulmonary hemorrhage. To the inventors' knowledge, acute pulmonary hemorrhage has not been reported previously in the veterinary literature, with the exception of exercise induced pulmonary hemorrhage in horses. The exact etiology of exercise-induced pulmonary hemorrhage is unknown, but it is presumed to be caused by hypertension associated with vigorous exercise. In humans, acute pulmonary hemorrhage can be associated with cardiac and vascular malformations, infectious or neoplastic processes, milk protein allergies, immune vasculitis, and trauma.

In humans, pulmonary hemorrhage has been associated with exposure to the mold S. chartarum, and pulmonary hemorrhage during anesthesia has been directly linked with exposure to S. chartarum. Seven months prior to anesthesia of the 2 cats described in the present report, the region had experienced a devastating hurricane with severe wind damage and flooding. On questioning, the owners of the 2 cats described in the present report indicated that their house did have minor flood damage as a result of the storm. Subsequent investigation revealed mold infiltration in their walls resulting from a roof leak that was so substantial as to require replacement of the walls.

Stachybotrys chartarum spores contain several classes of mycotoxins, most notably the trichothecene satratoxins G and H, which are potent protein synthesis inhibitors. In animals, these toxins appear to cause capillary fragility, which leads to pulmonary hemorrhage, and capillary fragility was the most likely cause of the pulmonary hemorrhage in the 2 cats described herein. Stachybotrys chartarum can also produce various phenylospirodrimanes, a cyclosporine, a hemolysin, and proteinases. In addition to their action as protein synthesis inhibitors, the trichothecenes are immunosuppressive, as are the phenylspirodrimanes and cyclosporine. Together, the trichothecenes and hemolysin produced by S chartarum can cause cell injury and death, leading to local destruction of the alveolar capillary wall. Collagen-degrading proteinases produced by Stachybotrys spores can possibly also cause acute structural damage to the alveolar capillary wall. As a result, the pulmonary capillaries would be at risk for stress failure whenever pulmonary capillary pressure or capillary transmural pressure was high.

Although cat 1 appeared to recover from the initial pulmonary hemorrhage that occurred during anesthesia, a second episode occurred the next day and the cat died. Cat 2 initially responded to treatment, but its condition deteriorated after the cat was discharged and returned to its contaminated environment.

Based upon the foregoing disclosure, it should now be apparent that exposure of an individual to a macrocyclic trichothecene can be determined by detecting a reaction of the macrocyclic trichothecene with a sample containing at least a portion of naturally occurring protein such as serum albumin. It is, therefore, to be understood that any variations evident fall within the scope of the claimed invention and thus, the selection of specific component elements can be determined without departing from the spirit of the invention herein disclosed and described.

The following abbreviations, as used herein, should be understood to have the following meanings:

ESI MS—electrospray ionization mass spectrometry;
HSA—human serum albumin;
MALDI-TOF MS—matrix assisted laser desorption/ionization time of flight mass spectroscopy;
MS—mass spectrometry;
PMF—peptide mass fingerprinting;
rHSA—recombinant human serum albumin;
S. chartarum—Stachybotrys chartarum;
SG—satratoxin G.

We claim:

1. A method of determining exposure of an individual to toxigenic Stachybotrys, the method comprising:
isolating a sample conatining at least a portion of a naturally occurring protein from an individual; and
detecting a reaction of a macrocyclic trichothecene with the at least a portion of a naturally occurring protein.

2. The method of claim 1, wherein detecting the reaction of a macrocyclic trichothecene comprises detecting the reaction of an antibody to an adduct of the macrocyclic trichothecene and at least a portion of a naturally occurring protein, wherein the antibody is raised against the macrocyclic trichothecene or an adduct thereof.

3. The method of claim 1, wherein the macrocyclic trichothecene is satratoxin G.

4. The method of claim 2, wherein the naturally occurring protein is selected from the group consisting of serum proteins.

5. The method of claim 2, wherein the protein is isolated from the blood of the individual.

6. The method of claim 1, wherein detecting the reaction of the macrocyclic trichothecene with the sample of at least a portion of naturally occurring protein comprises mass spectrometry.

7. The method of claim 2, wherein detecting the reaction of an antibody to an adduct of the macrocyclic trichothecene and the sample of at least a portion of a naturally occurring protein comprises affinity chromatography with the antibody.

8. A kit for determining the exposure of an individual to a macrocyclic trichothecene, the kit comprising an antibody that reacts to a macrocyclic trichothecene-protein adduct, wherein the antibody is capable of reacting to an adduct of at least a portion of a naturally occurring protein and the macrocyclic trichothecene.

9. The kit of claim 8, wherein the macrocyclic trichothecene is satratoxin G.

10. A method of determining exposure of an individual to a macrocyclic trichothecene, the method comprising:
isolating a sample of at least a portion of a naturally occurring protein from an individual;
reacting the sample of at least a portion of naturally occurring protein with an antibody that reacts to a macrocyclic trichothecene-protein adduct; and
detecting a reaction of the antibody with the sample of at least a portion of a naturally occurring protein.

11. The method of claim 10, wherein the naturally occurring protein is a blood protein.

12. The method of claim 11, wherein the macrocyclic trichothecene is satratoxin G.

13. The method of claim 10, wherein detecting the reaction of the antibody with the sample of at least a portion of a naturally occurring protein comprises mass spectrometry.

14. The method of claim 11, wherein the sample of at least a portion of a naturally occurring protein is the product of reacting a naturally occurring protein with a proteinase.

15. The method of claim 14, wherein the sample of at least a portion of a naturally occurring protein contains an amino acid capable of acting as a satratoxin adduct formation site, selected from the group consisting of lysine, histidine and cysteine.

16. The method of claim 11, wherein reacting the sample of at least a portion of naturally occurring protein with an antibody against a macrocyclic trichothecene and detecting a reaction of the antibody with the sample of at least a portion of naturally occurring protein is accomplished by an immunochemical method selected from the group consisting of enzyme linked immunosorbent assay, radioimmune assay and affinity chromatography.

17. A method of determining exposure of an individual to a macrocyclic trichothecene, the method comprising:
isolating a sample of at least a portion of a naturally occurring protein from an individual;
detecting a reaction of a macrocyclic trichothecene with the sample of at least a portion of a naturally occurring protein.

18. The method of claim 17, wherein detecting the reaction of the macrocyclic trichothecene with the sample of at least a portion of naturally occurring protein comprises mass spectrometry.

19. The method of claim 18, wherein the individual is a human.

20. The method of claim 18, wherein the individual is an animal.
